(19) United States
(12) Patent Application Publication
(10) Pub. No.: US 2015/0238399 A1
(43) Pub. Date: Aug. 27, 2015

(54) TOOTH WHITENING COMPOSITION

(71) Applicants: Matthew Scott Spaid, Amsterdam (NL); Petra Johanna Ruijs, Purmerend (NL)

(72) Inventors: Matthew Scott Spaid, Amsterdam (NL); Petra Johanna Ruijs, Purmerend (NL)

(21) Appl. No.: 14/429,747

(22) PCT Filed: Aug. 13, 2013
(86) PCT No.: PCT/NL2013/050597

§ 371 Date: Mar. 19, 2015

Related U.S. Application Data
(60) Provisional application No. 61/682,366, filed on Aug. 13, 2012.

(30) Foreign Application Priority Data
Sep. 21, 2012 (EP) 12185402.0

(51) Int. Cl. A61K 8/24; A61K 8/02; A61Q 11/00; A61K 8/22

(52) U.S. Cl. CPC A61K 8/24; A61K 8/22; A61K 8/0241; A61Q 11/00; A61K 2800/882; A61K 2800/413

(57) ABSTRACT

The present invention relates to a teeth whitening composition comprising 0.1 wt. % to 6 wt. % hydrogen peroxide, based on the total weight of the composition, and a nanohydroxy apatite having an average diameter of 0.1 to 20 nm. The present invention further relates to a teeth whitening kit comprising two separated components, wherein the first component comprises 0.1 wt. % to 6 wt. % hydrogen peroxide, based on the total weight of the first component, and the second component comprises a nanohydroxy apatite having an average diameter of 0.1 to 20 nm. The present invention also relates to a process for preparing a teeth whitening composition and to a dispenser for the teeth whitening kit.

### FIELD OF THE INVENTION

The present invention relates to a teeth whitening composition, in particular an in-office teeth whitening composition, comprising a low amount of hydrogen peroxide and a nanohydroxy apatite having an average diameter of 0.1 to 20 nm. The present invention further relates to a teeth whitening kit comprising two separated components, wherein the first component comprises the low amount of hydrogen peroxide and the second component comprises a nanohydroxy apatite having an average diameter of 0.1 to 20 nm. The present invention also relates to a process for preparing a teeth whitening composition. The present invention further relates to a dispenser for the teeth whitening kit, which enables a dental professional to adjust the mixing ratio of the first component and the second component.

### BACKGROUND OF THE INVENTION

This invention relates to a teeth whitening composition, preferably an in-office teeth whitening composition. In-office teeth whitening compositions are used by dental professionals. Various in-office teeth whitening systems are currently on the market. For example, the Zoom® whitening gel of Philips is applied to the teeth which is then activated by the Zoom light. The Dash® whitening system, also from Philips, is used without light activation and the product is said to contain 30 wt. % hydrogen peroxide. BriteSmile® of BriteSmile Inc. beaching gel contains 15 or 25 wt. % hydrogen peroxide. The product is applied to the teeth and then activated by blue light, i.e. the blue spectrum of visible light which has a wave length of about 380 to about 560 nm. The LaserSmile® teeth whitening system of Biolase Technology, Inc., operates a special laser. The Sapphire® whitening system of Ultradent Products Inc. and the Beyond® Power whitening system uses a gel containing 35% hydrogen peroxide. The LumaCool® whitening system uses light emitting diode technology in its in-office whitening system. The NUPRO® White Gold/Illumine™ professional teeth whitening system is manufactured by Dentsply International. Systems that are used without light activation must usually contain higher amounts of hydrogen peroxide to obtain the desired effect.

Instead of hydrogen peroxide, over the counter whitening products often contain carbamide peroxide.

U.S. Pat. No. 6,162,055, incorporated by reference, discloses a transparent tooth whitening composition comprising a photo-sensitizer, an oxidizing agent selected from peroxides and peroxyacids, e.g. hydrogen peroxide, carbamide peroxide, alkali metal peroxides, alkali metal percarbonates and alkali metal perborates. After application to teeth, the composition is activated with light having most preferably a wave length of 400 nm to 505 nm.

U.S. Pat. No. 6,368,765, incorporated by reference, discloses a teeth whitening composition comprising an oxidizing agent, preferably hydrogen peroxide, carbamide peroxide, benzoyl peroxide, glycerol peroxide, and sodium perborate, and potassium nitrate to reduce tooth sensitivity.

JP 8143436, incorporated by reference, discloses a bleaching agent comprising hydrogen peroxide, orthophosphoric acid and hydroxy apatite.

JP 9040539, incorporated by reference, discloses a dentifrice composition comprising 0.1 to 90 wt. % of a calcium compound, e.g. hydroxy apatite, and 0.05 to 10 wt. % carbamide peroxide. Carbamide peroxide has about one third of the strength of hydrogen peroxide. This implies that a 30 percent solution of carbamide peroxide is approximately equivalent of a 10 percent solution of hydrogen peroxide.

KR 100250920, incorporated by reference, discloses a toothpaste composition comprising a binding agent, a polishing agent, a whitening agent such as hydroxy apatite or hydrogen peroxide, a foaming agent and an enzyme.

KR 2002/0021446, incorporated by reference, discloses a teeth coating liquid comprising polyvinylpyrrolidone, a solvent selected from the group consisting of ethanol, water or a mixture thereof, tetra sodium pyrophosphate-H₂O₂, titanium dioxide and hydroxy apatite.

U.S. 2005/123490, incorporated by reference, discloses a tooth paste comprising hydroxyl apatite, hydrogen peroxide and phosphoric acid.

U.S. 2008/050408, incorporated by reference, discloses a teeth whitening composition comprising a calcium salt, e.g. a phosphate, and a source for peroxide. The composition may be foamed. The composition may be formulated in two or more components which are for example contained by a container comprising separate compartments.

WO 2011/161240, incorporated by reference, discloses a teeth whitening combined preparation comprising a first solution of a hydroxide and xylitol, a second solution of 15-35 wt. % hydrogen peroxide, and a third solution of a hydroxide, hydroxy apatite and xylitol. The preparation is used as follows: first the teeth are rinsed with the first solution and subsequently the second solution is applied to the teeth, where after the mouth is rinsed with water. This may be repeated one to five times. Finally, the mouth is rinsed with the third solution.

KR 2011/0129046, incorporated by reference, discloses a toothpaste composition comprising baking powder (which contains sodium hydrogen carbonate and potassium bitartrate) as an abrasive, D-sorbitol or glycerin as a sweetener, peppermint as a perfume, vitamin C as a nutrient, propylene glycol as a wetting agent, xanthan gum or gelatin as a binder, green tea or Glycyrrhizae radix extract as a cleansing agent, olive or olive leaf extract as a functional agent, sodium lauryl sulfate and hydrogen peroxide as a foaming agent, fluorine, polyethylene glycol 1500, sodium pyrophosphate and hydroxy apatite.

WO 00/03747, incorporated by reference, discloses apatite-based nanostructures materials wherein the crystallites have an average diameter of 0.5 to 200 nm. These materials may be used in odontostomatology.

WO 2005/020878, incorporated by reference, discloses a dental care product comprising a composite material containing poorly water soluble calcium salts in the form of nanoparticulate primary particles with a length of 5 to 150 nm and a cross-section of 2 to 50 nm.

Teeth whitening compositions are classified as cosmetic products under Annex III, Part 1, of the European Cosmetics Directive 76/768/EEC. The European Commission adopted new guidelines in September 2011 according to which products containing 0.1% to 6% hydrogen peroxide content may only be sold to dentists, and only under the conditions that (1) the first use of each cycle of the product must be completed by a dentist as a clinical examination, after which use may be continued by the patient and (2) the use of these products by clients under 18 years of age is not permitted, even under supervision of a dentist. Consequently, all tooth whitening products with hydrogen peroxide levels higher than 6% are to be banned in the European Union. These guidelines will be implemented in the new Cosmetic Regulation EC 1223/2009.

Consequently, there is a need in the art for effective teeth whitening compositions, in particular in-office teeth whitening compositions, comprising 6 wt. % hydrogen peroxide or less, in particular 0.1 wt. % to 6 wt. % hydrogen peroxide.

### SUMMARY OF THE INVENTION

The present invention relates to a teeth whitening composition comprising about 0.1% to about 6% hydrogen peroxide, based on the total weight of the composition, and a nanohydroxy apatite having an average diameter of about 0.1 to about 20 nm.

The present invention also relates to a teeth whitening kit comprising two separated components, wherein a first component comprises about 6 wt. % to about wt. 12% hydrogen peroxide, based on the total weight of the first component, and a second component comprises a nanohydroxy apatite having an average diameter of about 0.1 to about 20 nm.

The present invention further relates to a process for preparing a teeth whitening composition, wherein a first component and a second component are mixed in a ratio of between about 1:1 to about 1:120, the first component comprising about 6 wt. % to about 12 wt. % hydrogen peroxide, based on the total weight of the first component, and the second component comprising a nanohydroxy apatite having an average diameter of about 0.1 to about 20 nm.

The present invention further relates to a dispenser comprising at least two compartments, each compartment being provided with an outlet means, a first compartment comprising a first component and a second compartment comprising a second component, a mixing chamber connected to each outlet means, a manually adjustable adjustment means for adjusting a ratio of the first component and the second component supplied to the mixing chamber can be selected, the ratio being of between about 1:1 to about 1:120, wherein the first component comprises about 6 wt. % to about 12 wt. % hydrogen peroxide, based on the total weight of the first component, and the second component comprises a nanohydroxy apatite having an average diameter of about 0.1 to about 20 nm.

The present invention further relates to a method for whitening teeth, wherein a subject in need thereof is treated with a sufficient amount of a teeth whitening composition, said teeth whitening composition comprising about 0.1 wt. % to about 6 wt. % hydrogen peroxide, based on the total weight of the composition, and a nanohydroxy apatite having an average diameter of about 0.1 to about 20 nm, or said teeth whitening composition being obtainable by a process for preparing a teeth whitening composition, wherein a first component and a second component are mixed in a ratio of between about 1:1 to about 1:120, the first component comprising about 6 wt. % to about 12 wt. % hydrogen peroxide, based on the total weight of the first component, and the second component comprising a nanohydroxy apatite having an average diameter of about 0.1 to about 20 nm.

According to the present invention, it is preferred that the teeth whitening composition is an in-office teeth whitening composition.

### DETAILED DESCRIPTION OF THE INVENTION

The verb “to comprise” as is used in this description and in the claims and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

The term “nanohydroxy apatite” used in this document refers to materials having the chemical formula {Ca₁₀(PO₄)₆(OH)₂}. The average diameter of commercially available products may widely range, e.g. from about 0.1 to about 100 nm. The nanohydroxy apatite particles may have a needle-like, a fibroid or a spherical shape. Commercially available products are available in the form of powders and pastes. Manufactures include DFNano®, China, and Fluidinova (which commercialises nanohydroxy apatite under the trade name nanoXIM®), Portugal.

When the nanohydroxy apatite particles are non-spherical, the average diameter as defined in this document relates to the smallest diameter that can be assigned to the non-spherical particle. For example, the particle may have a needle-like structure with a length of about 100 nm and a diameter of e.g. about 10 nm. Such needle-like particles are therefore within the term “nanohydroxy apatite having an average diameter of about 0.1 to about 20 nm” as used in this document.

The term “teeth whitening” includes also cleaning of teeth. According to the present invention, the nanohydroxy apatite has preferably an average diameter of about 1 to about 20 nm, more preferably about 5 to about 20 nm, even more preferably about 5 to about 15 nm, and in particular about 7 to about 13 nm.

Preferably, the teeth whitening composition according to the present invention has an alkaline pH, preferably a pH of between more than about 7 to about 8.5. According to the present invention, the teeth whitening composition preferably comprises about 0.1 wt. % to 10 wt. % of the nanohydroxy apatite, based on the total weight of the composition, more preferably about 0.2 to about 7.5 wt. %.

According to the present invention, the teeth whitening composition comprises an acid, preferably an organic acid. Preferably, the acid is a phosphonic acid or a carboxylic acid and may therefore comprise one or more phosphonic acid groups and one or more carboxylic acid groups, respectively. The organic acid may comprise further functional groups, e.g. amine groups, hydroxy groups, and combinations thereof, and may therefore be a chelating organic acid. Suitable examples of organic acids include 1-hydroxyethylidene-1,1-diphosphonic acid (etidronic acid), ethylenediamine tetra(methylene phosphonic acid), acetic acid, maleic acid, 2-({2-[bis(carboxymethyl)amino]ethyl}(carboxymethyl)amino)acetic acid (EDTA) and the like. The organic acid may be present (in part or completely) in a salt form, preferably a salt of an alkali metal or an alkaline earth metal, and may therefore be a component of a buffer system. The alkali metal is preferably selected from the group consisting of lithium, sodium and potassium. The alkaline earth metal is preferably selected from the group consisting of magnesium or calcium.

It is preferred that the organic acid is a carboxlic acid comprising one or more carboxylic acid groups, preferable 1 to 6, more preferably 1 to 3 carboxylic acid groups. The carboxylic acid may optionally comprise one or more functional groups, preferably 1 to 3 functional groups, which are preferably amine groups, hydroxy groups, and combinations thereof. The carboxylic acid preferably comprises 2 to 12 carbon atoms, preferably 2 to 8 carbon atoms. The carboxylic acid is preferably aliphatic and may be linear or branched. Suitable carboxylic acids include acetic acid, citric acid, lactic acid, and oxalic acid.

It is preferred that the phosphonic acid is a phosphonic acid comprising one or more phosphonic acid groups, preferable 1 to 6, more preferably 1 to 3, even more preferably 1 or 2 phosphonic acid groups. The phosphonic acid may optionally comprise one or more functional groups, preferably 1 to 3 functional groups, which are preferably amine groups, hydroxy groups, and combinations thereof. The phosphonic acid preferably comprises 2 to 12 carbon atoms, preferably 2 to 8 carbon atoms. The phosphonic acid is preferably aliphatic and may be linear or branched. Suitable phosphonic acids include 1-hydroxyethylidene-1,1-diphosphonic acid (etidronic acid), ethylenediamine tetra(methylene phosphonic acid), (3-amino-l-hydroxypropane-1,1-diyl)bis(phosphonic acid) (pamidronic acid) and (4-amino-1-hydroxybutane-1,1-bis(phosphonic acid) (alendronic acid).

Preferably, the teeth whitening composition according to the present invention comprises a foaming agent. According to a preferred embodiment of the present invention, the foaming agent is a compound that produces carbon dioxide when it is contacted with an acid. The foaming agent is preferably inorganic, preferably a carbonate salt of an alkali metal or an alkaline earth metal, wherein the alkali metal is preferably selected from the group consisting of lithium, sodium and potassium, and wherein the alkaline earth metal is preferably be selected from the group consisting of magnesium or calcium. The foaming agent is preferably alkaline. A preferred example of a foaming agent is sodium hydrogen carbonate (NaHCO₃). According to the present invention, the teeth whitening composition preferably comprises about 0.1 wt. % to about 10 wt. % of a foaming agent, wherein the foaming agent is a compound that produces carbon dioxide when it is contacted with an acid.

According to another preferred embodiment of the present invention, the foaming agent is a surfactant, e.g. an anionic, cationic, ampholytic, non-ionic or zwitterionic surfactant. Preferably, the surfactant is an anionic surfactant, a non-ionic surfactant or a combination thereof. A preferred group of anionic surfactants is the group of C₈-C₂₄ alkyl sulphonates. A suitable example is sodium lauryl sulphonate. A preferred group of non-ionic surfactants is the group of poloxamers (Pluronic®) which are based on ethylene oxide, propylene oxide and combinations thereof. Such surfactants may also have wetting, dispersing, thickening, and emulsifying properties or combinations thereof. According to the present invention, the teeth whitening composition preferably comprises about 0.1 to about 10 wt. % of a foaming agent, wherein the foaming agent is a surfactant, preferably an anionic surfactant, a non-ionic surfactant or a combination thereof.

The teeth whitening composition according to the present invention may further comprise further components that are beneficial to teeth.

Preferably, the teeth whitening composition according to the present invention is in the form of a foam.

The present invention also relates to a teeth whitening kit comprising two separated components, wherein a first component comprises about 6 wt. % to about 12 wt. % hydrogen peroxide, based on the total weight of the first component, and a second component comprises a nanohydroxy apatite having an average diameter of about 0.1 to about 20 nm. Optionally, the first component may comprise about 6 wt. % to about 15 wt. % hydrogen peroxide, based on the total weight of the first component.

Preferably, the first component has a pH of about 2 to less than about 7, preferably about 2 to about 6, more preferably about 2 to about 5, and in particular about 2 to about 4. It is therefore preferred that the first component comprises an acid, preferably an organic acid. Preferably, the acid is a phosphonic acid, a carboxylic acid, or a combination thereof, and may therefore comprise one or more phosphonic acid groups and one or more carboxylic acid groups, respectively, as described above for carboxylic acids. The first component is in liquid form. The first component may further include usual ingredients commonly used in dental products such as thickeners and non-aqueous solvents.

Preferably, the second component has a pH of more than 7 to 11. It is therefore preferred that the second component comprises a base, preferably an inorganic base. Preferably, the base is selected from the hydroxides of the alkali metals.

The second component preferably comprises about 0.1 wt. % to about 10 wt. % of the nanohydroxy apatite, based on the total weight of the second component, more preferably about 0.2 wt. % to about 7.5 wt. %. The second component preferably further comprises a foaming agent, preferably in an amount of about 0.1 to about 10 wt. %, based on the total weight of the second component. The foaming agent is preferably a compound that produces carbon dioxide when it is contacted with an acid as is described above, a surfactant as described above, or a combination thereof.

The second component is in liquid form. The second component may further include usual ingredients commonly used in dental products such as thickeners and non-aqueous solvents.

The present invention also relates to a process for preparing a teeth whitening composition, wherein a first component and a second component are mixed in a ratio of between about 1:1 to about 1:120, the first component comprising about 6 wt. % to about 12 wt. % hydrogen peroxide, based on the total weight of the first component, and the second component comprising a nanohydroxy apatite having an average diameter of about 0.1 to about 20 nm. The present invention also relates to a teeth whitening composition obtainable by this process. Preferably, said teeth whitening composition comprises about 0.1 wt. % to about 6 wt. % hydrogen peroxide, based on the total weight of the composition, and a nanohydroxy apatite having an average diameter of about 0.1 to about 20 nm.

The present invention further relates to a dispenser comprising (a) at least two compartments, each compartment being provided with an outlet means, wherein a first compartment comprises a first component and a second compartment comprises a second component, (b) a mixing chamber connected to each outlet means, and (c) a manually adjustable adjustment means for adjusting or selecting a ratio of the first component and the second component supplied to the mixing chamber. The ratio is preferably between about 1:1 to about 1:120, wherein the first component preferably comprises about 6 wt. % to about 12 wt. % hydrogen peroxide, based on the total weight of the first component, and the second component preferably comprises a nanohydroxy apatite having an average diameter of 0.1 to 20 nm.

Accordingly, when the first component comprises about 12 wt. % hydrogen peroxide, it is mixed with the second component in a ratio of about 1:1 to obtain a teeth whitening composition comprising about 6 wt. % hydrogen peroxide. Similarly, when the first component comprises about 12 wt. % hydrogen peroxide, it is mixed with the second component in a ratio of about 1:120 to obtain a teeth whitening composition comprising about 0.1 wt. % hydrogen peroxide. When the first component comprises about 12 wt. % hydrogen peroxide and it is mixed with the second component in a ratio of about 1:2, a teeth whitening composition comprising about 3 wt. % hydrogen peroxide is obtained. Similarly, when the first component comprises about 6 wt. % hydrogen peroxide and it is mixed with the second component in a ratio of about 1:1, also a teeth whitening composition comprising about 3 wt. % hydrogen peroxide is obtained.

The teeth whitening composition is used in a method for whitening teeth. Accordingly, the present invention also provides a method for whitening teeth, wherein a subject in need thereof is treated with a sufficient amount of the teeth whitening composition according to the present invention. Accordingly, the present invention relates to a method for whitening teeth, said method comprising the treatment of teeth of a subject in need thereof with a teeth whitening composition comprising 0.1 wt. % to 6 wt. % hydrogen peroxide, based on the total weight of the composition, and a nanohydroxy apatite having an average diameter of 0.1 to 20 nm. This treatment is preferably conducted for about one to about twenty minutes, preferably for about five to about fifteen minutes and in particular for about ten minutes. After the teeth whitening composition is applied to the teeth, it is preferably activated by blue light. However, the teeth whitening composition can also be used without light activation.

The present invention also relates to a method for whitening teeth, wherein a subject in need thereof is treated with a sufficient amount of a teeth whitening composition, said teeth whitening composition being obtainable by a process for preparing a teeth whitening composition, wherein a first component and a second component are mixed in a ratio of between 1:1 to 1:120, the first component comprising 6 wt. % to 12 wt. % hydrogen peroxide, based on the total weight of the first component, and the second component comprising a nanohydroxy apatite having an average diameter of 0.1 to 20 nm.

The method for whitening teeth preferably comprises the following further steps. The first step of the method preferably comprises a polishing step. The second step of the method preferably comprises a pre-cleaning step. The third step of the method comprises the treatment with the teeth whitening composition according to the present invention. The fourth step preferably comprises a post-treatment step for enamel repair and reminiseralisation.

The polishing step is preferably conducted with usual dental equipment and materials.

The pre-cleaning step is preferably conducted with a pre-cleaning composition comprising about 0.1 wt. % to about 6 wt. % hydrogen peroxide, based on the total weight of the pre-cleaning composition. The series of steps involving the pre-cleaning step and the teeth whitening step is preferably repeated twice to ten times, more preferably three times to eight times, and most preferably four times to six times.

The post-treatment step is preferably conducted with an aqueous paste comprising about 5 to about 30 wt. % of nanohydroxyapatite having an average diameter of about 0.1 to about 50 nm, preferably about 0.1 to about 20 nm. Preferred pastes are nanoXIM® HAp102 and nanoXIM® HAp103 aqueous pastes of Fluidinova, Portugal. Preferably nanoXIM® HAp102 is used., The products nanoXIM® HAp102 and nanoXIM® HAp103 are hydroxyapatite nanoparticles aqueous pastes containing 15 wt. % and 30 wt. % nanohydroxy apatite particles having an average diameter of below 50 nm.

### EXAMPLES

The following examples further illustrate the preferred embodiments of the invention.

### Example 1

This example provides an example of the composition for a pre-treatment composition. This pre-treatment composition can be used prior to the treatment of the teeth whitening composition according to the present invention. Part A was mixed with Part B, where after Part C was added. Subsequently, Part D was added and the pH of the final mixture was adjusted to about 8.8.

| | Amount (wt. %) |
|---|---|
| Part A | |
| Water | 45.5 |
| KOH | 0.1 |
| Ionic surfactant^{a} | 2.0 |
| Anionic surfactant^{b} | 1.0 |
| Part B | |
| Water | 25.0 |
| EDTA | 0.2 |
| Sodium citrate | 0.9 |
| Part C | |
| H₂O₂ (35 wt. %) | 15.0 |
| Part D | |
| Ethanol | 10.0 |
| Tartaric acid | 0.05 |
| Fragrances | 0.25 |
| Total | 100 wt. % |

^{a}Combination of Poloxamer 188 and Poloxamer 407 (weight, ratio 1:1).
^{b}Sodium lauryl sulphate.

### Example 2

This example provides an example of the composition of the first component. Part B was mixed with Part A until both phases were fully dispersed. Then Part C was slowly added. The end-concentration of H₂O₂ is then 12 wt. %.

| | Amount (wt. %) |
|---|---|
| Part A | |
| Water | 52.0 |
| Glycerol | 10.0 |
| Xanthan gum | 0.7 |
| Part B | |
| Ethanol | 5.0 |
| Acetic acid | 1.0 |
| Citric acid | 5.0 |
| EDTA | 0.1 |
| Sodium citrate | 0.2 |
| Anionic surfactant^{a} | 1.0 |
| Non-ionic surfactant^{b} | 1.0 |
| Part C | |
| H₂O₂ (50%) | 24.0 |
| Total | 100 wt. % |

^{a}Sodium lauryl sulphate.
^{b}Pluronic P66.

### Example 3

This example provides an example of the composition of the second component. Part A was mixed with Part B, where after Part C was added.

| | Amount (wt. %) |
|---|---|
| Part A | |
| Water | 68.7 |
| Glycerol | 10 |
| Xanthan gum | 0.8 |
| Part B | |
| Nanohydroxy apatite paste | 1 |
| (aver. diam. 12 nm) | |
| Ethanol | 10 |
| KOH | 0.5 |
| Nonionic surfactant^{a} | 1 |
| Anionic surfactant^{b} | 2 |
| Part C | |
| NaHCO₃ | 4 |
| EDTA | 0.5 |
| Fragrances | 1.25 |
| Total | 100 wt. % |

^{a}Poloxamer 68.
^{b}Sodium lauryl sulphate.

### Example 4

The efficacy of the teeth whitening composition according to Examples 2 and 3 was tested on patients. First, the tooth shade colour of the patients was determined by using the Vita Shade Guide (Vident®, Brea, Calif., USA). The VITA Classical shade guide consists of 16 shades (in value order: B1, A1, B2, D2, A2, Cl, C2, D4, A3, D3, B3, A3.5, B4, C3, A4, C4). For bleached teeth, The VITA Bleached Shades which consist of shades 0M1, 0M2 and 0M3 from the VITA 3D-Master Shade System are used. The order of bleached shades from lightest to darkest is: M1 0, M1 0.5, M1 1, then B1, A1, B2, etc.

The patients were instructed to brush and floss their teeth. Severe surface stains were then removed from the teeth. The teeth were then pre-treated with the pre-treatment composition according to Example 1 by applying the composition with a cotton swab. The first and second components according to Examples 2 and 3 were mixed at a ratio of 1:1 so that a teeth whitening composition of 6 wt. % hydrogen peroxide is obtained in the form of a foam. This composition was then applied to the teeth by using a brush. In the next step, the composition was activated by blue light for ten minutes. The composition was then removed by suction. The series of the pre-treatment step to the suction step was repeated six times (for more stained teeth, the series of the pre-treatment step to the suction step may be repeated eight times). After the sixth time, the teeth were rinsed with water. In the last step, the teeth were treated with nanoXIM® HAp102 which was applied by using a sponge or a cotton swab. The product nanoXIM® HAp102 is commercially available from Fluidinova, Portugal, and it is a paste containing 15 wt. % nanohydroxy apatite particles having an average diameter of below 50 nm. The whitened teeth were then evaluated for the tooth shade colour. The results obtained with 11 patients are summarised below.

| | Vita shade^{a}(before - | Vita shade^{a} | Improvement of |
|---|---|---|---|
| Patient No. | darkest teeth) | (after) | Shades |
| 1 | A3.5 | A1 | 11 |
| 2 | A1 | M1 0 | 4 |
| 3 | B2 | 0.5 M1 | 4 |
| 4 | A3.5 | A1 | 10 |
| 5 | A3 | 0.5 M1 | 10 |
| 6 | B2 | 0.5 M1 | 4 |
| 7 | A3 | 1 M1 | 9 |
| 8 | A3 | 1 M1 | 9 |
| 9 | A3.5 | A1 | 10 |
| 10 | A3.5 | B1 | 11 |
| 11 | A3 | A1 | 10 |
| 12 | B4 | A1 | 11 |

^{a}determined on the bicuspids or darkest shade.

These data show that the treatment with the teeth whitening composition provides an improvement of four to eleven shades of the Vita Classic and Bleaching Shade Guide. This improvement is comparable with improvements claimed by manufactures of currently commercially available products containing 15 to 35 wt. % hydrogen peroxide.

1. A teeth whitening composition comprising 0.1 wt. % to 6 wt. % hydrogen peroxide, based on the total weight of the composition, and a nanohydroxy apatite having an average diameter of 0.1 to 20 nm.

2. The teeth whitening composition according to claim 1, having a pH of between more than 7 to 8.5.

3. The teeth whitening composition according to claim 1, comprising 0.1 wt. % to 10 wt. % of the nanohydroxy apatite, based on the total weight of the composition.

4. The teeth whitening composition according to claim 1, further comprising an acid.

5. The teeth whitening composition according to claim 1, further comprising a foaming agent.

6. A teeth whitening kit comprising two separated components, wherein a first component comprises 6 wt. % to 12 wt. % hydrogen peroxide, based on the total weight of the first component, and a second component comprises a nanohydroxy apatite having an average diameter of 0.1 to 20 nm.

7. The teeth whitening kit according to claim 6, wherein the first component has a pH of 2 to less than 7.

8. The teeth whitening kit according to claim 6, wherein the first component further comprises an acid.

9. The teeth whitening kit according to claim 6, wherein the second component has a pH between 7 to 11.

10. The teeth whitening kit according to claim 6, wherein the second component further comprises a base selected from the hydroxides of the alkali metals.

11. The teeth whitening kit according to claim 6, wherein the second component comprises 0.1 wt. % to 10 wt. % of the nanohydroxy apatite, based on the total weight of the second component.

12. The teeth whitening kit according to claim 6, wherein the second component further comprises a foaming agent.

13. A process for preparing a teeth whitening composition, comprising mixing a first component and a second component in a ratio between 1:1 to 1:120, the first component comprising 6 wt. % to 12 wt. % hydrogen peroxide, based on the total weight of the first component, and the second component comprising a nanohydroxy apatite having an average diameter of 0.1 to 20 nm.

14. A teeth whitening composition obtainable by the process of claim 13.

15. The teeth whitening composition according to claim 14, comprising 0.1 wt. % to 6 wt. % hydrogen peroxide, based on the total weight of the composition, and a nanohydroxy apatite having an average diameter of 0.1 to 20 nm.

16. A dispenser comprising at least two compartments, each compartment being provided with an outlet means, a first compartment comprising a first component and a second compartment comprising a second component, a mixing chamber connected to each outlet means, a manually adjustable adjustment means for adjusting or selecting a ratio of the first component and the second component supplied to the mixing chamber, the ratio being of between 1:1 to 1:120, wherein the first component comprises 6 wt. % to 12 wt. % hydrogen peroxide, based on the total weight of the first component, and the second component comprises a nanohydroxy apatite having an average diameter of 0.1 to 20 nm.

17. A method of whitening teeth, comprising administering to a subject in need thereof a sufficient amount of a teeth whitening composition comprising 0.1 wt. % to 6 wt. % hydrogen peroxide, based on the total weight of the composition, and a nanohydroxy apatite having an average diameter of 0.1 to 20 nm.
